(19) United States
(12) Patent Application Publication
(10) Pub. No.: US 2023/0024366 A1
(43) Pub. Date: Jan. 26, 2023

(54) SYSTEMS AND METHODS FOR SERIOUS ILLNESS IDENTIFICATION AND STRATIFICATION

(71) Applicant: Cambia Health Solutions, Inc., Portland, OR (US)

(72) Inventors: April Lynn Krutka, Park City, UT (US); Jennifer Alexis Tschirpke, Portland, OR (US); Kathryn Jones Rees, Portland, OR (US)

(21) Appl. No.: 17/813,798

(22) Filed: Jul. 20, 2022

Related U.S. Application Data
(60) Provisional application No. 63/223,877, filed on Jul. 20, 2021.

(51) Int. Cl. G16H 50/30; G16H 50/20; G16H 50/70; G16H 10/60

(52) U.S. Cl. CPC G16H 50/30; G16H 50/20; G16H 50/70; G16H 10/60

(57) ABSTRACT

Systems and methods for serious illness identification and stratification are provided. In one embodiment, a method includes calculating functional scores in a plurality of categories based on healthcare data for an individual, calculating a total serious illness score for the individual based on the functional scores, measuring a serious illness score change for the individual based on the total serious illness score and historical serious illness scores for the individual, updating a clinical queue of a plurality of clinical queues to include the individual based on the serious illness score change and the total serious illness score, wherein each clinical queue comprises a list of individuals prioritized for healthcare intervention, and transmitting the updated clinical queue to a client device associated with a clinician. In this way, healthcare data may be leveraged to characterize the health status of an individual over time, and to prioritize serious illness care accordingly.

### CROSS REFERENCE TO RELATED APPLICATIONS

The present application claims priority to U.S. Provisional Application No. 63/223,877 entitled “SYSTEMS AND METHOD FOR SERIOUS ILLNESS IDENTIFICATION AND STRATIFICATION” and filed on Jul. 20, 2021. The entire contents of the above-identified application are hereby incorporated by reference for all purposes.

### FIELD

The present description relates generally to health care management, and in particular to evaluating risk of serious illness for individuals.

### BACKGROUND AND SUMMARY

Despite recent advancements in the ability to perform predictive modeling and risk stratification to identify high-risk individuals, a significant amount of time may pass before a clinician, such as a nurse, may contact or otherwise intervene with an individual to provide proactive care. In some instances, the individual may undergo hospitalization or suffer from a medical episode before without ever being identified as a patient in need. Further, current risk models for serious illness often demand a person to have a poor prognosis or acute utilization of healthcare services. Serious illness may herein be defined as a health condition that carries a high risk of mortality and either negatively impacts a person's daily function or quality of life, or excessively strains their caregivers. The focus on prognosis or acute utilization severely limits many people that have needs demanding proactive support. This makes the task of providing proactive support through a population health strategy impossible, and creates unnecessary suffering for patients and their caregivers. This perpetuates a legacy healthcare model of crisis management in acute care settings.

In addition, healthcare costs are a significant portion of the United States Gross National Product, and continue to rise. A significant portion of these expenses represent costs attributed to individuals who utilize health care services to a higher degree than average. As a minority of healthcare users generate the majority of healthcare costs, predictive modeling of the overall population of healthcare consumers may be effectively used to identify such healthcare users. Customized education and clinical support may then be offered to identified healthcare users to help individuals address their health and avoid expensive medical events in the future. By proactively monitoring and improving the health of the minority of the population that consumes the majority of healthcare resources, many preventable medical expenses such as hospitalization and emergency room visits may be significantly reduced.

The inventors have recognized the above issues and have devised several approaches to address them. In particular, systems and methods for serious illness identification and stratification are provided. In one embodiment, a method comprises storing healthcare data for a population of individuals in a plurality of network-based databases according to a plurality of categories, converting the healthcare data to functional scores of the plurality of categories for each individual of the population of individuals, calculating a total serious illness score for each individual based on the functional scores and storing the total serious illness score at a serious illness score database of the serious illness score module, retrieving the total serious illness score and historical illness scores for an individual of the population of individuals from the serious illness score database, in response to a request for a serious illness assessment of the individual, and determining a serious illness score change for the individual based on the total serious illness score and historical serious illness scores for the individual, automatically assigning the individual to a clinical queue of a plurality of clinical queues based on the serious illness score change and the total serious illness score, and automatically updating the clinical queue to include the individual, wherein each clinical queue comprises a list of individuals prioritized for healthcare intervention, automatically generating, a message comprising the updated clinical queue, and transmitting the message to at least one client device associated with a clinician to allow the clinician to review the updated clinical queue, and provide recommendations for treatment based on the updated clinical queue.

In this way, experience and physical function data is leveraged to characterize the health status of an individual over time, and to prioritize interventions accordingly. Further, by processing healthcare data in this way, an entire serious illness population may be stratified, thereby allowing personalized interventions at each stage of disease through a population health strategy.

The above summary is provided to introduce a selection of concepts in a simplified form that are further described below in the Detailed Description. This summary is not intended to identify key features or essential features of the subject matter, nor is it intended to be used to limit the scope of the subject matter. Furthermore, the subject matter is not limited to implementations that solve any or all of the disadvantages noted above or in any part of this disclosure.

### BRIEF DESCRIPTION OF THE FIGURES

FIG. 1 shows a block diagram of an example computing system for evaluating risk of serious illness in individuals, according to an embodiment.

FIG. 2 shows a block diagram illustrating an example module architecture for evaluating risk of serious illness in individuals, according to an embodiment.

FIG. 3 shows a graph illustrating example health function trajectories for various serious illnesses, according to an embodiment.

FIG. 4 shows a set of graphs illustrating example calculations of a serious illness score over time for an individual, according to an embodiment.

FIG. 5 shows a high-level flow chart illustrating an example method for evaluating serious illness scores for individuals to identify and stratify risk of serious illness for the individuals, according to an embodiment.

FIG. 6 shows a high-level flow chart illustrating an example method for queueing individuals based on serious illness scores to prioritize healthcare interventions, according to an embodiment.

FIG. 7 shows a set of graphs illustrating example changes in serious illness scores over time for different serious illnesses, according to an embodiment.

FIG. 8 shows a graph illustrating an example queueing timeline for an individual, according to an embodiment.

### DETAILED DESCRIPTION

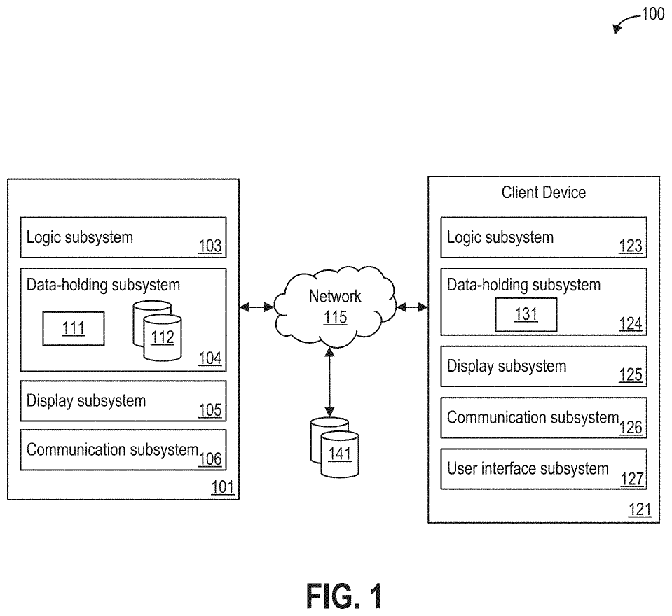

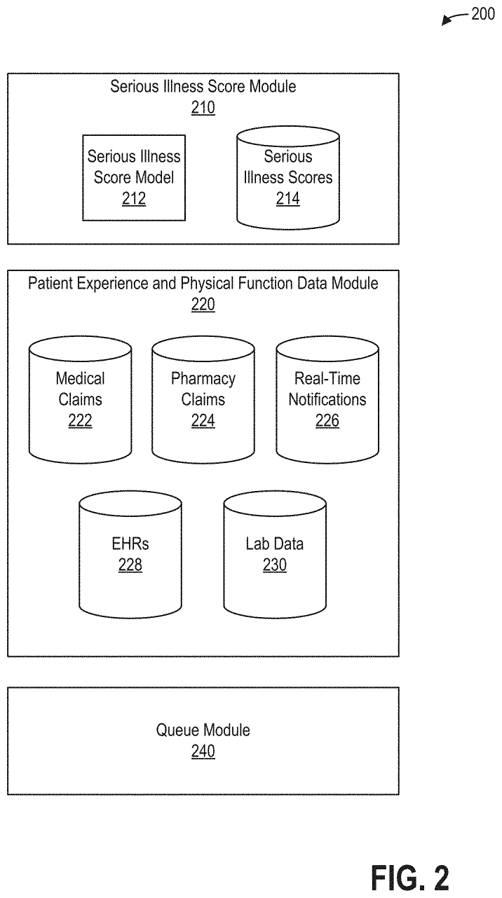

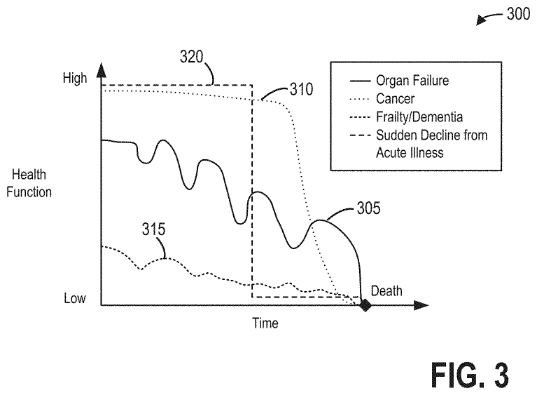

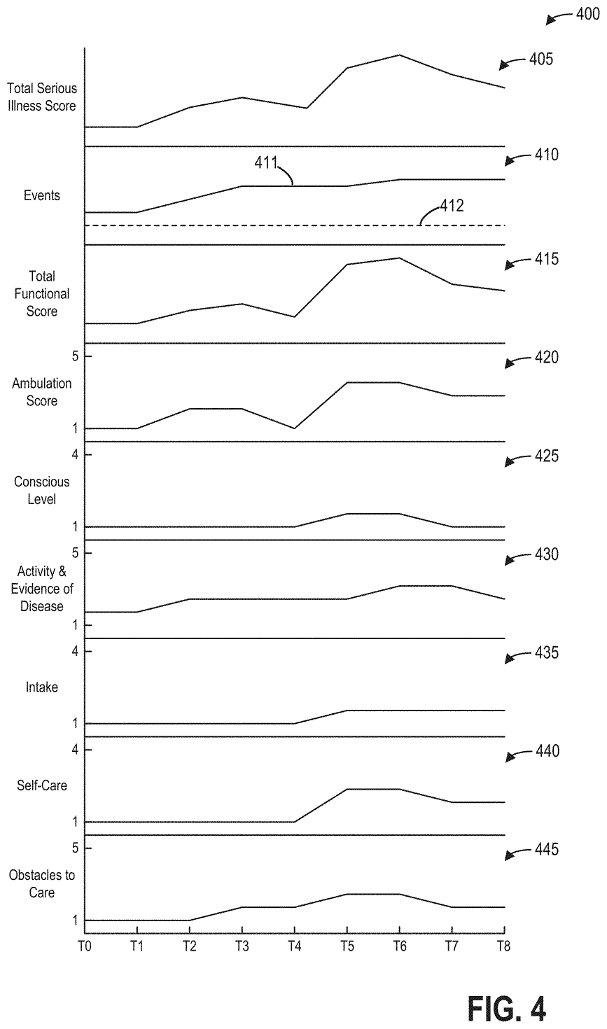

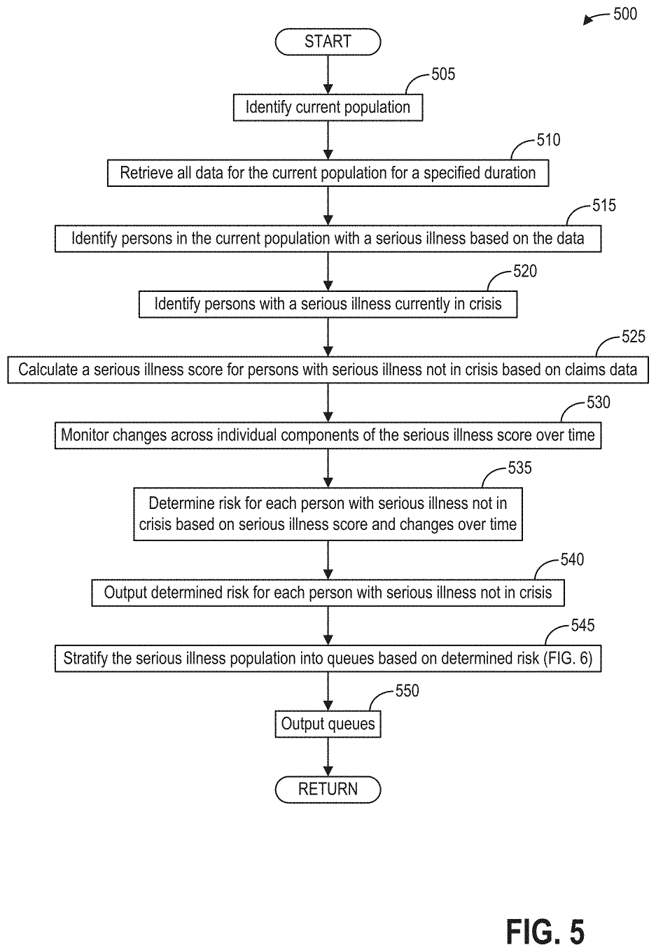

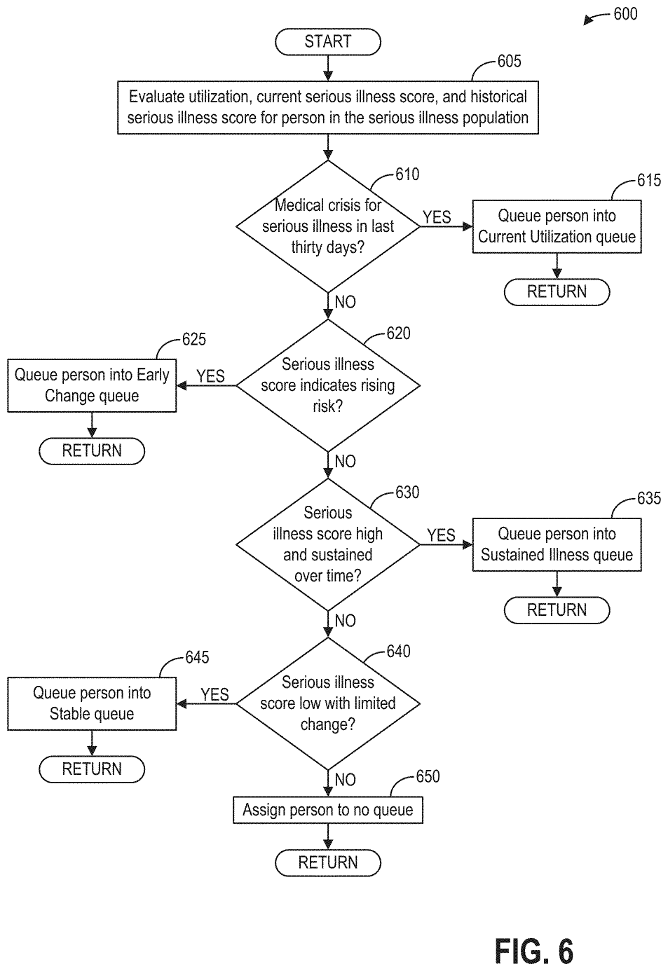

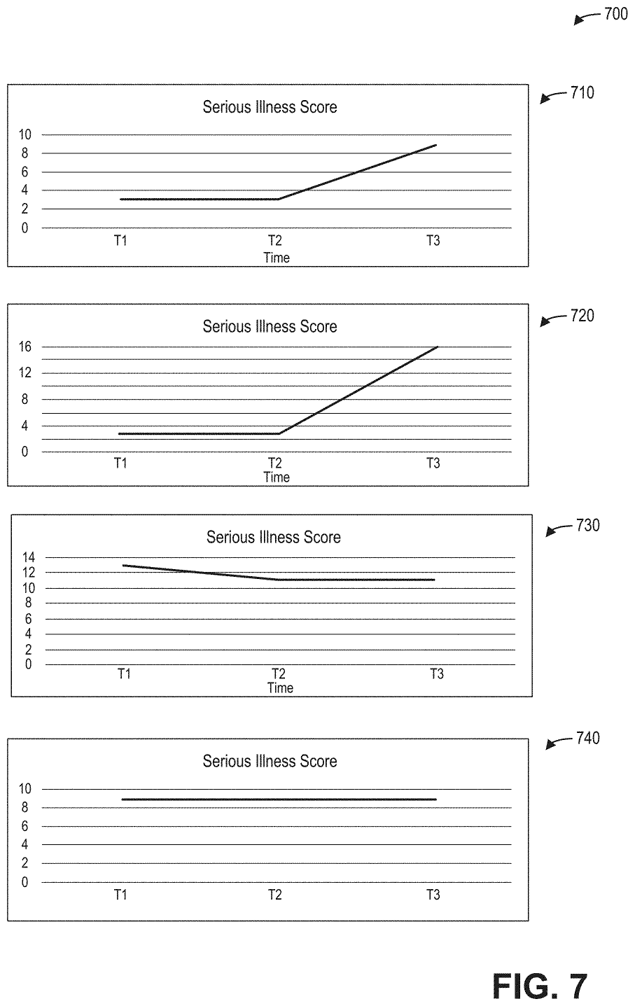

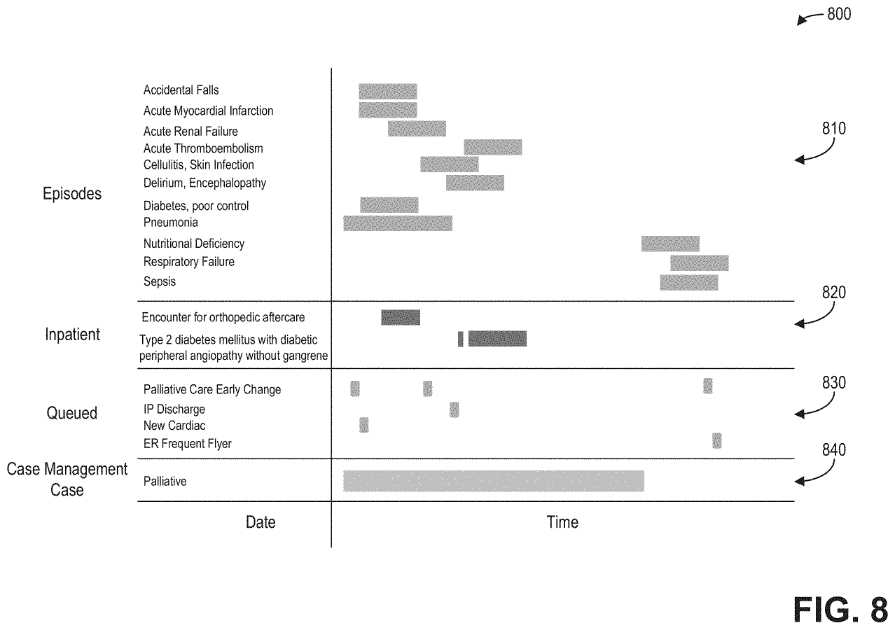

The present description relates to systems and methods for serious illness identification and stratification. In particular, systems and methods are provided for measuring a serious illness score based on healthcare data for individuals in a serious illness population. A computing environment, such as the computer environment or system depicted in FIG. 1, may include a healthcare management system which consolidates healthcare data for a plurality of individuals from a disparate and heterogeneous plurality of data sources, derives measures of different health functions from the healthcare data for the plurality of individuals, calculates a total serious illness score based on the measures of different health functions, and stratifies the plurality of individuals according to the total serious illness score for each individual. Such a healthcare management system, as depicted in FIG. 2, may include modules configured to aggregate disparate data in different formats, selectively extract data from the disparate data to determine measures of functional health that characterize the health status of individuals, and generate queues for healthcare interventions according to the measures of functional health over time. By evaluating health function over time, individuals with serious illness with less drastic health trajectories, as depicted in FIG. 3, may nevertheless be identified and scheduled for palliative care interventions. The total serious illness score over time may be measured from a plurality of functional scores as well as different health-related events, as depicted in FIG. 4. By tracking the individual experience and functional scores over time, particular contributing factors to declining health status may be identified such that care interventions may be personalized. A method for serious illness risk stratification, such as the method shown in FIG. 5, may include identifying individuals in a serious illness population not currently in crisis, and calculating serious illness scores for such individuals based on healthcare data such as medical claims. According to utilization and serious illness scores over time, each individual in population may be assigned to queues, as shown in FIG. 6. Examples of serious illness trends that may correspond to different risks are shown in FIG. 7. An example timeline for serious illness interventions and queues for an individual experiencing a plurality of health episodes over time is shown in FIG. 8.

Turning now to the figures, FIG. 1 illustrates an example computing environment 100 in accordance with the current disclosure. In particular, computing environment 100 includes a server 101, a plurality of user devices or client systems including at least one client device 121, and a network 115. However, not all of the components illustrated in FIG. 1 may be required to practice the present disclosure. Variations in the arrangement and type of the components may be made without departing from the spirit or scope of the invention.

Server 101 may be a computing device configured to evaluate healthcare data obtained from heterogeneous healthcare data sources to identify risk of serious illness in individuals. In different embodiments, server 101 may take the form of a mainframe computer, server computer, desktop computer, laptop computer, tablet computer, network computing device, mobile computing device, mobile communication device, and so on.

Server 101 may include a logic subsystem 103 and a data-holding subsystem 104. Server 101 may optionally include a display subsystem 105, communication subsystem 106, and/or other components not shown in FIG. 1. For example, server 101 may also optionally include user input devices such as keyboards, mice, game controllers, cameras, microphones, and/or touch screens.

Logic subsystem 103 may include one or more physical devices configured to execute one or more instructions. For example, logic subsystem 103 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

As one example, logic subsystem 103 may include one or more processors that are configured to execute software instructions. Additionally or alternatively, the logic subsystem 103 may include one or more hardware or firmware logic machines configured to execute hardware or firmware instructions. Processors of the logic subsystem 103 may be single or multi-core computer processors, and the programs executed thereon may be configured for parallel or distributed processing. The logic subsystem 103 may optionally include individual components that are distributed throughout two or more devices, which may be remotely located and/or configured for coordinated processing. One or more aspects of the logic subsystem 103 may be virtualized and executed by remotely accessible networked computing devices configured in a cloud computing configuration.

Data-holding subsystem 104 may include one or more physical, non-transitory memory devices configured to hold data and/or instructions executable by the logic subsystem 103 to implement the herein described methods and processes. When such methods and processes are implemented, the state of data-holding subsystem 104 may be transformed (for example, to hold different data).

In one example, the server 101 includes a healthcare management system 111 configured as executable instructions in the data-holding subsystem 104. The healthcare management system 111 may evaluate healthcare data from a plurality of healthcare data sources to measure risk of serious illness in a plurality of individuals and to coordinate the provision of care to such individuals. To that end, one or more local healthcare databases 112 comprising aggregated healthcare data from a plurality of healthcare data sources may be stored in the data-holding subsystem 104 and accessible to the healthcare management system 111. As discussed further herein, the one or more local healthcare databases 112 may include a database of serious illness scores measured for individuals in order to track changes in serious illness scores over time. The one or more healthcare databases may further include healthcare data and other data stored locally (e.g., one or more local healthcare databases 112) or remotely, for example in one or more network-based healthcare databases 141 communicatively coupled to the server 101 via the network 115.

Data-holding subsystem 104 may include removable media and/or built-in devices. Data-holding subsystem 104 may include optical memory (for example, CD, DVD, HD-DVD, Blu-Ray Disc, etc.), and/or magnetic memory devices (for example, hard drive disk, floppy disk drive, tape drive, MRAM, etc.), and the like. Data-holding subsystem 104 may include devices with one or more of the following characteristics: volatile, nonvolatile, dynamic, static, read/write, read-only, random access, sequential access, location addressable, file addressable, and content addressable. In some embodiments, logic subsystem 103 and data-holding subsystem 104 may be integrated into one or more common devices, such as an application-specific integrated circuit or a system on a chip.

It is to be appreciated that data-holding subsystem 104 includes one or more physical, non-transitory devices. In contrast, in some embodiments aspects of the instructions described herein may be propagated in a transitory fashion by a pure signal (for example, an electromagnetic signal) that is not held by a physical device for at least a finite duration. Furthermore, data and/or other forms of information pertaining to the present disclosure may be propagated by a pure signal.

When included, display subsystem 105 may be used to present a visual representation of data held by data-holding subsystem 104. As the herein described methods and processes change the data held by the data-holding subsystem 104, and thus transform the state of the data-holding subsystem 104, the state of display subsystem 105 may likewise be transformed to visually represent changes in the underlying data. Display subsystem 105 may include one or more display devices utilizing virtually any type of technology. Such display devices may be combined with logic subsystem 103 and/or data-holding subsystem 104 in a shared enclosure, or such display devices may be peripheral display devices.

When included, communication subsystem 106 may be configured to communicatively couple server 101 with one or more other computing devices, such as client device 121. Communication subsystem 106 may include wired and/or wireless communication devices compatible with one or more different communication protocols. As non-limiting examples, communication subsystem 106 may be configured for communication via a wireless telephone network, a wireless local area network, a wired local area network, a wireless wide area network, a wired wide area network, etc. In some embodiments, communication subsystem 106 may allow server 101 to send and/or receive messages to and/or from other devices via a network such as the public Internet. For example, communication subsystem 106 may communicatively couple server 101 with client device 121 via network 115. In some examples, network 115 may be the public Internet. In other examples, network 115 may be regarded as a private network connection and may include, for example, a virtual private network or an encryption or other security mechanism employed over the public Internet.

Further, the server 101 provides a network service that is accessible to a plurality of users through a plurality of client systems such as the client device 121 communicatively coupled to the server 101 via the network 115. As such, computing environment 100 may include one or more devices operated by users, such as client device 121. Client device 121 may be any computing device configured to access a network such as network 115, including but not limited to a personal desktop computer, a laptop, a smartphone, a tablet, and the like. While one client device 121 is shown, it may be appreciated that any number of user devices may be communicatively coupled to the server 101 via the network 115.

Client device 121 includes a logic subsystem 123 and a data-holding subsystem 124. Client device 121 may optionally include a display subsystem 125, communication subsystem 126, a user interface subsystem 127, and/or other components not shown in FIG. 1.

Logic subsystem 123 may include one or more physical devices configured to execute one or more instructions. For example, logic subsystem 123 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

As one example, logic subsystem 123 may include one or more processors that are configured to execute software instructions. Additionally or alternatively, the logic subsystem 123 may include one or more hardware or firmware logic machines configured to execute hardware or firmware instructions. Processors of the logic subsystem 123 may be single or multi-core, and the programs executed thereon may be configured for parallel or distributed processing. The logic subsystem 123 may optionally include individual components that are distributed throughout two or more devices, which may be remotely located and/or configured for coordinated processing. One or more aspects of the logic subsystem 123 may be virtualized and executed by remotely accessible networking computing devices configured in a cloud computing configuration.

Data-holding subsystem 124 may include one or more physical, non-transitory memory devices configured to hold data and/or instructions executable by the logic subsystem 123 to implement the herein described methods and processes. When such methods and processes are implemented, the state of data-holding subsystem 124 may be transformed (for example, to hold different data).

Data-holding subsystem 124 may include removable media and/or built-in devices.

Data-holding subsystem 124 may include optical memory (for example, CD, DVD, HD-DVD, Blu-Ray Disc, etc.), and/or magnetic memory devices (for example, hard drive disk, floppy disk drive, tape drive, MRAM, etc.), and the like. Data-holding subsystem 124 may include devices with one or more of the following characteristics: volatile, nonvolatile, dynamic, static, read/write, read-only, random access, sequential access, location addressable, file addressable, and content addressable. In some embodiments, logic subsystem 123 and data-holding subsystem 124 may be integrated into one or more common devices, such as an application-specific integrated circuit or a system on a chip.

When included, display subsystem 125 may be used to present a visual representation of data held by data-holding subsystem 124. As the herein described methods and processes change the data held by the data-holding subsystem 124, and thus transform the state of the data-holding subsystem 124, the state of display subsystem 125 may likewise be transformed to visually represent changes in the underlying data. Display subsystem 125 may include one or more display devices utilizing virtually any type of technology. Such display devices may be combined with logic subsystem 123 and/or data-holding subsystem 124 in a shared enclosure, or such display devices may be peripheral display devices.

In one example, the client device 121 may include executable instructions 131 in the data-holding subsystem 124 that when executed by the logic subsystem 123 cause the logic subsystem 123 to perform various actions as described further herein. As one example, the client device 121 may be configured, via the instructions 131, to receive one or more serious illness scores for one or more patients from the server 101, and display the one or more serious illness scores for the one or more patients via a graphical user interface on the display subsystem 125 to a user such as a healthcare provider. The client device 121 may be further configured to receive feedback regarding the one or more serious illness scores via the user interface subsystem 127, and transmit the feedback to the server 101 for updating the one or more serious illness scores based on the feedback.

When included, communication subsystem 126 may be configured to communicatively couple client device 121 with one or more other computing devices, such as server 101. Communication subsystem 126 may include wired and/or wireless communication devices compatible with one or more different communication protocols. As non-limiting examples, communication subsystem 126 may be configured for communication via a wireless telephone network, a wireless local area network, a wired local area network, a wireless wide area network, a wired wide area network, etc. In some embodiments, communication subsystem 126 may allow client device 121 to send and/or receive messages to and/or from other devices, such as server 101, via a network 115 such as the public Internet.

Client device 121 may further include a user interface subsystem 127 comprising user input devices such as keyboards, mice, game controllers, cameras, microphones, and/or touch screens. A user of client device 121 may input feedback regarding a serious illness score for one or more individuals, for example, via user interface subsystem 127. As discussed further herein, client device 121 may transmit, via communication subsystem 126, user input received via the user interface subsystem 127 to the server 101 over the network 115. In this way, the server 101 may update, based on the user feedback, one or more serious illness scores for one or more individuals and/or a serious illness score model configured to calculate the one or more serious illness scores.

Thus server 101 and client device 121 may each represent computing devices which may generally include any device that is configured to perform computation and that is capable of sending and receiving data communications by way of one or more wired and/or wireless communication interfaces. Such devices may be configured to communicate using any of a variety of network protocols. For example, client device 121 may be configured to execute a browser application that employs HTTP to request information from server 101 and then displays the retrieved information to a user on a display such as the display subsystem 125.

FIG. 2 shows an overview of an exemplary arrangement of software modules for a healthcare management system 200 configured to calculate serious illness scores for members. The healthcare management system 200 may be implemented, as an illustrative example, as executable instructions of the healthcare management system 111 in the data-holding subsystem 104 of a server 101 of FIG. 1 above. The healthcare management system 200 comprises a plurality of modules, including but not limited to a serious illness score module 210, a patient experience and physical function data module 220, and a queue module 240. The modules depicted are exemplary, and in some examples the healthcare management system 200 may include different combinations of modules including or not including the modules depicted in FIG. 2 as described further herein.

The serious illness score module 210 may be configured to calculate serious illness scores for individuals based on healthcare data. The serious illness score module 210 may include a serious illness score model 212 configured to calculate a serious illness score for an individual based on healthcare data for the individual obtained via the patient experience and physical function data module 220, for example, and a serious illness score database 214 configured to store calculations of serious illness scores for individuals over time, as an illustrative and non-limiting example.

The patient experience and physical function data module 220 aggregates data relating to one or more patients from a plurality of disparate data sources and manages storage of such aggregated data for use by the serious illness score module 210. As an illustrative and non-limiting example, the patient experience and physical function data module 220, also referred to herein simply as the patient data module 220, may include a plurality of databases including a medical claims database 222 storing medical claims for individuals, a pharmacy claims database 224 storing pharmacy claims for individuals, a real-time notifications database 226 storing real-time notifications relating to care events for individuals, an electronic health records (EHRs) database 228 storing EHRs for individuals, and a lab data database 230 storing laboratory data for individuals.

The queue module 240 may be configured to generate one or more queues for clinicians comprising a plurality of patients or individuals organized and prioritized for healthcare interventions based on the serious illness score(s) (e.g., from the serious illness score database 214) for each patient of the plurality of patients.

FIG. 3 shows a graph 300 illustrating example health function trajectories for various serious illnesses, as determined in Comstock Barker P, Scherer JS. Illness Trajectories: Description and Clinical Use #326. J Palliat Med. 2017; 20(4):426-427. The graph 300 includes a health trajectory 305 for a patient with organ failure, a health trajectory 310 for a patient with cancer, a health trajectory 315 for a patient with frailty/dementia, and a health trajectory 320 for a patient with a sudden decline from an acute illness. As depicted, the health function of patients with different serious illnesses ranges from high health function to low health function over time until the serious illness results in death, but the health trajectories vary depending on the type of serious illness. For example, the health trajectories 310 and 320 for cancer and sudden decline from acute illness, respectively, both exhibit a steep downward slope from high function to low function, whereas the health trajectories 305 and 315 for organ failure and frailty/dementia, respectively, both exhibit a relatively lower downward slope with semi-periodic swings between high and low function over time as indicated by the peaks and valleys of the health trajectories 305 and 315.

FIG. 4 shows a set of graphs 400 illustrating example calculations of a serious illness score over time for an individual, according to an embodiment. In particular, the graphs 400 include a graph 405 depicting a total serious illness score over time, a graph 410 depicting relevant event scores (e.g., historical health event scores) over time, a graph 415 depicting a total functional score over time, a graph 420 depicting an ambulation function score over time, a graph 425 depicting a conscious level function score over time, a graph 430 depicting an activity-and-evidence-of-disease function score over time, a graph 435 depicting an intake function score over time, a graph 440 depicting a self-care function score over time, and a graph 445 depicting an obstacles-to-care function score over time.

The total serious illness score illustrated by graph 405 may comprise a sum of the event (e.g., health event) scores depicted in graph 410 and the total functional score depicted in graph 415. The graph 410 in particular depicts a plot 411 of key events relating to critical care as well as a plot 412 of ongoing events. The plot 411 measures key historical events such as admission to critical care which may be permanently counted in the total serious illness score, in some examples. Such key historical events comprise medical events that make a person vulnerable and are included in the total serious illness score. The value of the plot 411 may therefore increase or stay fixed over time and may not decrease. The plot 412 measures ongoing events that may be temporary in duration, and thus may only be counted towards the total serious illness score when such events are occurring. Ongoing events may include, but are not limited to, infections, falls, inpatient admissions, outpatient emergency room visits, and so on. The value of the plot 412 may thus comprise a count of specific events indicating a health status change over time, and so the value of the plot 412 may increase, stay fixed, or decrease over time.

The total functional score depicted in graph 415 may include a sum of each functional score, such as the function scores depicted in the graphs 420, 425, 430, 435, 440, and 445 as an illustrative and non-limiting example. In other words, the total functional score may be a compilation of information provided by the individual function scores and trends in the total functional score over time may be identified based on graph 415.

The ambulation function score depicted in graph 420 may include a measure of ambulation of the person, where ambulation comprises the ability of the person to walk without assistance. As an illustrative and non-limiting example, the ambulation function score may range from a value of one to five, where a value of one indicates the person has full independent mobility while a value of five indicates the person is bed bound and immobile. Values between one and five may correspond to the use of different ambulatory assistance devices, such as walkers, canes, crutches, wheelchairs, gait belts, and so on. The ambulation function score may therefore be determined based on healthcare data indicating the use of such ambulatory assistance devices or otherwise specifying the mobility of the person, such as medical claims, prescriptions for ambulatory assistance devices, and/or an EHR for the person.

The conscious level function score depicted in graph 425 may comprise a measure of a conscious level of the person. As an illustrative and non-limiting example, the conscious level function score may range from a value of one to four, where a value of one indicates the person is fully alert while a value of four indicates the person is comatose, with values therebetween indicating a decline in conscious level. The conscious level function score may therefore be determined based on healthcare data indicating the conscious level of the person, such as medical claims data and/or an EHR for the person.

The activity-and-evidence-of-disease function score depicted in graph 430 may comprise a measure of disease activity and evidence of a disease. As an illustrative and non-limiting example, the activity-and-evidence-of-disease function score may range from a value of one to five, where a value of one indicates no disease and normal activity while a value of five indicates extensive evidence of disease and an inability to perform daily activities, with values therebetween indicating intermediate stages between normal activity and an inability to perform daily activities. The activity-and-evidence-of-disease function score may be determined based on healthcare data indicating the evidence of disease and consequent activity, such as diagnostic codes in medical claims data for the person and/or EHRs.

The intake function score depicted in graph 435 may comprise a measure of food intake ability for the person. As an illustrative and non-limiting example, the intake function score may range from a value of one to four, where a value of one indicates a normal ability for food intake while a value of four indicates a reliance on feeding tubes, with values therebetween indicating a corresponding dependence on assistance for food intake. The intake function score may be determined based on one or more of healthcare data indicating the food intake ability for the person, such as medical claims, pharmacy claims, EHRs, and so on.

The self-care function score depicted in graph 440 may comprise a measure of an ability for the person to care for themselves without assistance. As an illustrative and non-limiting example, the self-care function score may comprise a value ranging from one to four, where a value of one indicates a full self-care ability while a value of four indicates that total care is required, with values therebetween indicating a decline in ability for self-care. The self-care function score may be determined based on healthcare data indicating the ability for the person to care for themselves, such as based on EHRs and/or medical claims.

The obstacles-to-care function score depicted in graph 445 may comprise a measure of obstacles to care for the person. As an illustrative and non-limiting example, the obstacles-to-care function score may range from a value of one to five, where a value of one indicates that all needs are met for the person while a value of five indicates that total care is required, with values therebetween indicating an increase in obstacles to care. The obstacles-to-care function score may be determined based on healthcare data indicating obstacles to care, such as EHRs and/or medical claims.

In the example depicted in the set graphs 400, the function scores are measured at each time point (e.g., at the times T0, T1, T2, T3, T4, T5, T6, T7, and T8) which may comprise a fixed interval or a dynamic interval. For example, function scores may be calculated for a person responsive to a patient data module (such as patient data module 220 of FIG. 2) receiving new and/or updated healthcare data relating to the person. When the patient data module receives new or updated healthcare data relating to the person, for example, a serious illness score module (such as serious illness score module 210 of FIG. 2) may evaluate the new or updated healthcare data to determine whether to update one or more function scores based on the new or updated healthcare data. It may be appreciated that one or more function scores may be not be updated based on new or updated healthcare data while other function scores may be updated based on the new or updated healthcare data. In other examples, the serious illness score module may calculate function scores based on all current healthcare data relating to a person on a schedule, such as on a daily basis or at selected intervals (e.g., monthly, every forty-five days, and so on as illustrative and non-limiting examples), regardless of whether new or updated healthcare data relating to the person is provided to the patient data module.

As such, the total serious illness score may be determined for a patient for a current period of care and may be compared with historical serious illness scores. Changes in the total serious illness score may be observed over time and trends in the changes identified, allowing a future condition of the patient to be predicted efficiently and accurately. Information provided by determining the total serious illness score may present a healthcare provider with a robust and streamlined method for assessing and predicting the patient's health status based on large quantities of data. The healthcare provider may therefore be better informed for recommending treatment for the patient.

FIG. 5 shows a high-level flow chart illustrating an example method 500 for evaluating serious illness scores for individuals to identify and stratify risk of serious illness for the individuals, according to an embodiment. In particular, method 500 relates to calculating serious illness scores for persons based on healthcare claims data and queueing the persons into queues based on the serious illness scores. Method 500 is described with regard to the systems and components of FIGS. 1 and 2, though it may be appreciated that the method 500 may be implemented with other systems and components without departing from the scope of the present disclosure. Method 500 may be implemented as executable instructions (e.g., an executable program) in a non-transitory computer-readable storage medium, such as the data-holding subsystem 104, and may be executed by a processor, such as the logic subsystem 103.

Method 500 begins at 505. A user (e.g., clinician) may initiate method 500 to prioritize healthcare needs of a population. The user may wish to efficiently identify any patients which may benefit from a healthcare intervention given a finite amount of time and resources. At 505, method 500 identifies a current population to be evaluated as described herein. The current population may be identified based on demographics such as age, geographic location, association with a given health insurance plan, healthcare facility, and/or healthcare provider, other factors, or combinations thereof. For example, method 500 may identify a current population comprising persons in a given demographic and geographic region associated with a particular health insurance plan. As another illustrative and non-limiting example, method 500 may identify a current population comprising all persons associated with a particular health insurance plan. The number of persons in the current population may comprise thousands or even millions of individuals.

At 510, method 500 includes retrieving all data for the current population for a specified duration. For example, all healthcare data for each person may be retrieved in the current population generated or uploaded within the specified duration prior to executing method 500. As an illustrative example, the specified duration may comprise one day, and so all data for the current population may be retrieved that was uploaded or generated within the past day. As another illustrative example, the specified duration may comprise one month, and so all data for the current population may be retrieved that was uploaded or generated within the past month. As yet another illustrative example, the specified duration may comprise one year, and so all data for the current population may be retrieved that was uploaded or generated within the past year. As an illustrative and non-limiting example, for a current eligible population of two million individuals, the data may comprise upwards of sixty million medical claims amounting to approximately one-hundred million claim lines, ten million pharmacy claims, and hundreds of thousands of real-time admission, discharge, and emergency room notifications. The data may further include electronic health records and laboratory data.

At 515, method 500 includes identifying persons in the current population with a serious illness based on the data. For example, a serious illness population within the current population may be identified based on diagnosis codes indicating a serious illness in the medical claims data. In this way, the number of individuals to evaluate may be decreased from two million to just over one-hundred thousand individuals, as an illustrative example. The diagnosis codes used to identify the serious illness population may include diagnosis codes associated with organ failure such as congestive heart failure (CHF), chronic obstructive pulmonary disease (COPD), and chronic kidney disease (CKD), neurologic or frailty diseases such as dementia, Parkinson's, or amyotrophic lateral sclerosis (ALS), and cancer, as illustrative and non-limiting examples.

At 520, persons with a serious illness currently in crisis are identified. For example, method 500 identifies persons in the serious illness population who are currently in crisis by identifying persons who are currently in an inpatient setting or in an emergency room for their serious illness. In some examples, method 500 may identify such persons currently in crisis due to a serious illness based on a combination of real-time notifications (e.g., real-time admission, discharge, and transfer data) and medical claims data. Additionally or alternatively, method 500 may further identify persons currently in crisis due to a serious illness based on a combination of diagnosis codes, procedure codes, place-of-service codes, and revenue codes in medical claims data to identify such persons based on claims data alone with or without real-time notifications data. By identifying persons with serious illness currently in crisis, such persons may be prioritized for providing coordinated care, ordering palliative consults, and ensuring that such persons' needs are met.

At 525, method 500 includes calculating a serious illness score for persons with serious illness not in crisis based on the claims data. That is, all serious illness population members not in crisis may be assessed to identify their current risk level across various components. To that end, method 500 includes assigning scores for a functional status in a plurality of categories for each person based on converting one or more claim lines and other healthcare data to function scores. The functional categories may include, as illustrative and non-limiting examples, ambulation, conscious level, activity and evidence of disease, intake, self-care, and obstacles to care.

Ambulation examples may include bed confinement, use of a walker, and so on, so the healthcare data for a person may be processed to identify diagnosis codes, procedure codes, and so on associated with such ambulation levels to assign an ambulation function score. The ambulation function score may range from one to five, where one indicates full mobility, two indicates reduced mobility, three indicates the person mainly sits or lies, four indicates the person is mainly in bed, and five indicates the person is completely bed-bound.

Conscious level examples include an altered mental state and brain anoxia, and so the healthcare data for a person may be processed to identify claims lines associated with such conscious levels to assign a conscious level function score. The conscious level function score may range from one to four, where one indicates the person has a full conscious level, two indicates the person is fully conscious with some confusion, three indicates the person is fully conscious with confusion or drowsy, and four indicates the person is drowsy or comatose.

Examples of activity and evidence of disease may include incontinence and dialysis, and so the healthcare data for a person may be processed to identify diagnosis codes and procedure codes that indicate evidence of disease, and assign an activity-and-evidence-of-disease function score based on such evidence of disease. The activity-and-evidence-of-disease function score may range from one to five, where one indicates normal activity and no evidence of disease, two indicates normal activity with effort and some evidence of disease, three indicates significant disease evidence and that the person is unable to do normal work or other activity, four indicates extensive evidence of disease and that the person is unable to do most activities, and five indicates extensive evidence of disease and that the person is unable to do any daily activities.

Examples of intake may include lack of appetite or anorexia, and the use of feeding tubes, so method 500 may include processing the healthcare data for a person to identify medical claim lines or other evidence indicating declined food intake, and assign the intake function score based on the identified evidence of declined food intake. The intake function score may range from one to four, where one indicates normal food intake, two indicates reduced food intake, three indicates minimal food intake or sips, and four indicates reliance on feeding tubes.

Self-care examples may include therapy for bathing or toileting and colostomy, and so the healthcare data for a person may be processed to identify evidence of declined self-care ability, and assign a self-care function score based on any identified evidence. The self-care function score may range from one to four, for example, where one indicates full self-care ability, two indicates occasional assistance, three indicates considerable assistance, and four indicates total care.

Examples of obstacles to care may include housing status, economic status, and social isolation, and so method 500 may include processing the healthcare data for a person to identify any evidence of such obstacles to care, and assign an obstacles-to-care function score based on any identified evidence. The obstacles-to-care function score may range from one to five, for example where one indicates that all needs are met while five indicates that the person is struggling with basic physiological needs.

Method 500 may further include assigning one or more event scores, including an ongoing event score and a key event score. The ongoing event score may comprise a count of events that indicate a changing health status over time, where examples of ongoing events include infections, falls, inpatient stays, and emergency room visits. The key event score may comprise a historical count of admissions to critical care. In this way, for people with otherwise identical functional scores and ongoing events, a person who has experienced one or more previous admissions to critical care may have a higher serious illness score than a person without any previous admissions to critical care.

At 530, method 500 includes monitoring changes across individual components of the serious illness score over time for each person. For example, a change in each component of the serious illness score may be calculated, including the total serious illness score, relative to at least one previous calculation of the serious illness score. The serious illness score may be compared to a plurality of previous calculations of the serious illness score, for example, in order to track changes and trends over time.

At 535, method 500 includes determining risk for each person with serious illness not in crisis based on the serious illness score and changes over time. As an illustrative and non-limiting example, method 500 may include categorizing a risk for each person based on the serious illness score and changes over time. For example, the risk for each person may be categorized as rising risk, sustained illness, or stable. A person may be categorized as rising risk if the person is experiencing a significant change in status and is at risk for adverse outcomes, indicating by changes above a delta threshold (e.g., predetermined to indicate a significant change in health status) combined with the current serious illness score. The changes over time may be positive (e.g., increasing) rather than negative (e.g., decreasing). For example, a person with a change greater than the delta threshold to a serious illness score above a score threshold may be categorized as rising risk. A person may be categorized as sustained illness if the serious illness score is fairly stable over time (e.g., changes over time are less than the delta threshold) while the baseline serious illness score is higher. For this risk category, ambulatory care may be meeting the needs of the person, but if the person were to experience an illness or exacerbation of their disease, they are more likely to experience a medical crisis. A person may be categorized as stable if the person is not experiencing major changes in their illness (e.g., changes over time are less than the delta threshold) and their serious illness score is below a score threshold. The risk categories thus correspond to ranges of serious illness scores and ranges of serious illness score changes over time, separated by one or more score thresholds and delta thresholds. Method 500 may further include calculating a quantitative risk value based on the serious illness scores and changes over time in order to prioritize persons within a given risk category. For example, some individuals categorized with rising risk may have a relatively higher risk than other individuals categorized with rising risk.

At 540, method 500 includes outputting the determined risk for each person with serious illness not in crisis. For example, method 500 may output a serious illness assessment including the determined risk to a data-holding subsystem (such as data-holding subsystem 104 of FIG. 1) for storage. Outputting the determined risk may include outputting the serious illness score to a serious illness score database (such as serious illness score database 214 of FIG. 2), as an illustrative example.

At 545, method 500 includes stratifying the serious illness population into queues based on the determined risk. For example, via a queue module (such as queue module 240 of FIG. 2) as an example, each person in the serious illness population may be added to a queue corresponding to the determined risk. Each person may be assigned a position in a queue according to the serious illness score, for example. An example method for queueing individuals in the serious illness population is described further herein with regard to FIG. 6.

At 550, method 500 includes outputting the queues. Method 500 may output the queues to one or more client devices, such as the client device 121 of FIG. 1, via a network such as network 115 of FIG. 1, such that a healthcare provider or another person managing healthcare cases may review the queues and associated recommendations and act accordingly. For example, a user of the client device receiving the queues may contact one or more individuals in the queues to schedule an appointment or otherwise provide a healthcare intervention appropriate for the determined risk. Method 500 then returns.

FIG. 6 shows a high-level flow chart illustrating an example method 600 for queueing individuals based on serious illness scores to prioritize healthcare interventions, according to an embodiment. In particular, method 600 relates to assigning persons in a serious illness population to queues based on the serious illness scores. Method 600 is described with regard to the systems and components of FIGS. 1 and 2, though it may be appreciated that the method 600 may be implemented with other systems and components without departing from the scope of the present disclosure. Method 600 may be implemented as executable instructions (e.g., an executable program) in a non-transitory computer-readable storage medium, such as the data-holding subsystem 104 of FIG. 1, and may be executed by a processor, such as the logic subsystem 103 of FIG. 1. As an example, method 600 may be implemented by the queue module 240 of the healthcare management system 200 as described with respect to FIG. 2 in the server 101 of FIG. 1.

Method 600 begins at 605. At 605, method 600 includes evaluating the utilization, current serious illness score, and historical serious illness score for a person in the serious illness population. For example, the utilization for the person may be evaluated based on real-time notifications, medical claims, and/or EHRs for the person to determine whether the person is currently utilizing healthcare services (e.g., if the person is currently admitted into inpatient care). Method 600 further including evaluating the current serious illness score for the person relative to historical serious illness scores, for example to determine changes in serious illness scores over time.

At 610, method 600 includes determining whether the person has experienced a medical crisis for a serious illness in the last thirty days. The person may be determined to have experienced a medical crisis for a serious illness in the last thirty days based on utilization. For example, if real-time notifications, medical claims, and/or EHRs for the person indicate admission and/or discharge or other utilization within the last thirty days in connection to the serious illness, method 600 includes determining that the person has experienced a medical crisis for the serious illness in the last thirty days. If the person has experienced a medical crisis for a serious illness in the last thirty days (“YES”), method 600 continues to 615. At 615, method 600 includes queuing the person into a “Current Utilization” queue. Method 600 then returns.

However, referring again to 610, if the person has not experienced a medical crisis for a serious illness in the last thirty days (“NO”), method 600 continues to 620. At 620, method 600 includes determining whether the serious illness score for the person indicates a rising risk. Method 600 may include determining that the person has a rising risk if the person has a utilization claim within a certain time duration (e.g., the prior forty-five days), a current serious illness score above a score threshold (e.g., indicating a relatively higher risk), and a change in serious illness score over time above a delta threshold (e.g., indicating that a decline in functionality for the person), as these factors combined indicate that the person may be headed towards a medical crisis. If the serious illness score indicates a rising risk (“YES”), method 600 continues to 625. At 625, method 600 includes queuing the person into an “Early Change” queue. Method 600 then returns.

However, referring again to 620, if the serious illness score does not indicate a rising risk (“NO”), method 600 continues to 630. At 630, method 600 includes determining whether the serious illness score is high and sustained over time. The serious illness score may be determined to be high and sustained over time, for example, if the person has a utilization claim within a certain time duration (e.g., the prior forty-five days), a current serious illness score above the score threshold, and a change in serious illness score over time is less than the delta threshold, as these factors indicate that the person is “seriously sick” where the person has a heavy burden of illness but has possibly adapted and their health is relatively stable. If the serious illness score is high and sustained over time (“YES”), method 600 proceeds to 635. At 635, method 600 includes queuing the person into a “Sustained Illness” queue. Method 600 then returns.

However, referring again to 630, if the serious illness score is not high and sustained over time (“NO”), method 600 continues to 640. At 640, method 600 includes determining whether the serious illness score is low with limited change. The serious illness score may be determined to be low with limited change, for example, if the person has a utilization claim within the certain time duration (e.g., the prior forty-five days), a current serious illness score below the score threshold, and a change in serious illness score over time less than the delta threshold, as these factors indicate that the person is living relatively well with a serious illness, with stable health and ambulatory care meeting their needs. If the serious illness score is low with limited change (“YES”), method 600 continues to 645. At 645, method 600 includes queuing the person into a “Stable” queue. Method 600 then returns.

However, referring again to 640, if the serious illness score is not low with limited change (“NO”), method 600 continues to 650. At 650, method 600 includes assigning the person to no queue. Alternatively, method 600 may include queuing the person into a “Review” queue for subsequent review of their serious illness score measures and confirmation of their health by a care provider. Method 600 then returns.

As illustrative and non-limiting examples of how individuals may be assigned to different queues based on serious illness scores, FIG. 7 shows a set of graphs 700 illustrating example changes in serious illness scores over time for different serious illnesses, according to an embodiment. For example, graph 710 illustrates example serious illness scores over time for a person with COPD and chronic respiratory failure. During a first time period T1 prior to evaluating the current serious illness score, the person has a total serious illness score of three. The first time period T1 may comprise an adjustable temporal duration, and may comprise a time duration on the order of minutes or hours (e.g., for ongoing evaluation of a person's score), a day (e.g., for daily monitoring), to weeks or months (e.g., for monitoring longer term effects), to years (e.g., for monitoring health over a lifetime). As an illustrative and non-limiting example, each time period including the first time period T1 may comprise a duration of forty-five days, such that the first time period T1 corresponds to a period of 90-135 days prior to the current evaluation of the serious illness score. As an illustrative example, the person may have a functional score of three due to a cough and use of oxygen at home during the first time period T1. During a second time period T2 prior to evaluating the current serious illness score, the person may have a total serious illness score of three, for example due to the use of home oxygen. During a third time period T3 comprising a most recent period of evaluating the current serious illness score, the person has a total serious illness score of nine, for example due to three ongoing events (e.g., corresponding to three infections) as well as six functional points (e.g., due to contributions from anorexia, cough, home oxygen, infections, severe pneumonia, and shortness of breath). As the serious illness score changes from three to nine, the change in total serious illness score by six may be above a delta threshold and the current serious illness score of nine may be above a score threshold. Consequently, the person may be assigned to an “Early Change” queue as discussed hereinabove.

As another illustrative example, the graph 720 illustrates example serious illness scores for a person with heart failure. During the first time period, the person has a total serious illness score of three, for example due to one key event (e.g., a single historical admission to critical care) and two palliative or functional points (e.g., due to weakness). During the second time period, the person has a total serious illness score of three, for example due to the one key event which is permanently counted in the total serious illness score, as well as two ongoing events (e.g., due to a fall). Although the point contributors are different, the total serious illness score is the same. During the third time period, the person has a total serious illness score of sixteen, for example due to the one key event, four ongoing events (e.g., four falls within the time period), and eleven functional points (e.g., due to an altered mental state, confusion, encephalopathy, housing status, abdomen pain, back pain, syncope, and weakness). As the total serious illness score increases by thirteen, the change in serious illness score may be above the delta threshold and the current total serious illness score may be above the score threshold. Consequently, the person may be assigned to the “Early Change” queue.

As another illustrative example, the graph 730 illustrates example serious illness scores over time for a person with chronic respiratory failure. During the first time period T1, the person has a total serious illness score of thirteen, for example due to one key event (e.g., admission to critical care), and twelve functional points (e.g., due to gait, home oxygen, tubes, ventilator, and weakness identified from claims data). During the second time period T2, the person has a total serious illness score of eleven, for example due to the one key event and ten functional points (e.g., corresponding to a hemorrhage, home oxygen, tubes, ventilator, and weakness). During the third time period T3, the person has a total serious illness score of eleven, for example due to the one key event and ten functional points (e.g., corresponding to use of home oxygen, tubes, ventilator, and weakness). Although the total serious illness score decreases from the first time period T1 to the second time period T2 and does not indicate any change from the second time period T2 to the third time period T3, such that the change over time is below a delta threshold, the total serious illness score is above a score threshold. Consequently, the person may be assigned to a “Sustained Illness” queue.

As yet another illustrative example, the graph 740 illustrates example serious illness scores over time for a person with COPD, chronic respiratory failure, and an inflammatory lung disease. During the first period T1, the person has a total serious illness score of nine, for example due to one key event (e.g., historical critical care admission), one ongoing event (e.g., an infection), and seven functional points (e.g., due to gait, home oxygen, tubes, ventilator, and weakness). During the second period T2, the person has a total serious illness score of nine, for example due to the one key event, the one ongoing event, and seven functional points (e.g., hemorrhage, home oxygen, tubes, ventilator, and weakness). During the third time period T3, the person has a total serious illness score of nine, for example due to the one key event, the ongoing event, and seven functional points (e.g., due to home oxygen, tubes, ventilator, and weakness). Although the point contributors to the functional points may change over time, the relative severity of one or more point contributors may change and so the total functional score may be consistent over time. As the change over time is below a delta threshold while the total serious illness score is above a score threshold, the person may be assigned to a “Sustained Illness” queue. Further, by tracking point contributors and changes in individual function scores over time, a clinician evaluating the historical serious illness score may quickly glean how the health status of the person over time has stabilized while also understanding what factors contributed to their sustained health status.

FIG. 8 shows a graph 800 illustrating an example of a queueing timeline for an individual, according to an embodiment. The graph 800 includes a plot 810 of different medical episodes experienced by the individual over time, a plot 820 of in-patient treatment received by the individual over time, a plot 830 of queues the individual is assigned to over time, and a plot 840 indicating a case management case for palliative care for the individual. The individual may be identified for palliative care based on the queues of the individual as shown in plot 830, where the queues may be assigned and updated automatically following methods described above with respect to FIGS. 5-6. Further, the individual may be automatically identified for palliative care for a duration of time as shown by plot 840. Absent of a method for automatically queuing and assigning the individual for a level and/or type of palliative care, the patient may not receive suitable treatment mitigating subsequent drastic changes in health conditions that may otherwise impose strain on a healthcare system.

The technical effect of calculating serious illness scores and queuing individuals based on their score, as well as the change in their score over time, is that an otherwise prohibitively large set of data may be managed in a manner that enables efficient extraction of requested data and automatic conversion of the data into a comprehensive display presented to a user, e.g., a healthcare provider. By storing the data, e.g., healthcare data, in databases according to a type of the data, the different types of data may be readily retrieved and analyzed to convert the data into functional scores, the functional scores corresponding to a plurality of categories of the healthcare data and generated based on compiling information across the plurality of categories according to points in time. Converting the data into the functional scores allows changes in a serious illness score to be observed, thereby enabling identification of change that may be indicative of a demand for palliative care that may otherwise be difficult to determine based on large quantities of datasets stored across different databases. In some instances, by processing the data as described herein, early detection of serious illness may be accomplished prior to overt physical manifestation of the serious illness. Such early detection may be challenging for the healthcare provider to achieve without the categorization and analysis of data provided by the methods described herein. Furthermore, automatic updating of the data, and of resulting serious illness queues generated based on the data, allows messages displayed to the healthcare provider, e.g., messages relaying the serious illness queues at a client device of the healthcare provider, to maintain a high level of accuracy over time.

The disclosure also provides support for a method, comprising: storing healthcare data for a population of individuals in a plurality of network-based databases according to a plurality of categories, converting, with a processor configured with a serious illness score module, the healthcare data to functional scores of the plurality of categories for each individual of the population of individuals, calculating, with the processor, a total serious illness score for each individual based on the functional scores and storing the total serious illness score at a serious illness score database of the serious illness score module, retrieving, with the processor, the total serious illness score and historical serious illness scores for an individual of the population of individuals from the serious illness score database, in response to a request for a serious illness assessment of the individual, and determining a serious illness score change for the individual based on the total serious illness score and historical serious illness scores for the individual, automatically assigning, with a queue module of the processor, the individual to a clinical queue of a plurality of clinical queues based on the serious illness score change and the total serious illness score, and automatically updating the clinical queue to include the individual, wherein each clinical queue comprises a list of individuals prioritized for healthcare intervention, automatically generating, with the queue module, a message comprising the updated clinical queue, and transmitting, with the processor, the message to at least one client device associated with a clinician to allow the clinician to review the updated clinical queue and provide recommendations for treatment based on the updated clinical queue. In a first example of the method, converting the healthcare data to the functional scores of the plurality of categories comprises processing medical claims for the population of individuals associated with a time duration to identify one or more claim lines, and generating a functional score for a category of the plurality of categories based on the one or more claim lines. In a second example of the method, optionally including the first example, the method further comprises: generating the functional score for the category based on the one or more claim lines according to a model configured to convert the one or more claim lines to the functional score for the category. In a third example of the method, optionally including one or both of the first and second examples, determining the serious illness score change comprises measuring changes in each functional score of the functional scores over time for the individual. In a fourth example of the method, optionally including one or more or each of the first through third examples, the method further comprises: calculating event scores for the individual based on ongoing health events and historical health events, and calculating the total serious illness score based on the event scores and the functional scores. In a fifth example of the method, optionally including one or more or each of the first through fourth examples, the plurality of categories includes ambulation, conscious level, activity and evidence of disease, food intake, self-care ability, and obstacles to care. In a sixth example of the method, optionally including one or more or each of the first through fifth examples, automatically updating the clinical queue to include the individual based on the serious illness score change and the total serious illness score includes assigning the individual to a position within the clinical queue based on the total serious illness score.

The disclosure also provides support for a computer-readable storage medium including an executable program stored thereon, the executable program configured to cause a computer processor to: store healthcare data for a population of individuals in a plurality of network-based databases according to a plurality of categories, convert the healthcare data to functional scores of the plurality of categories for each individual of the population of individuals, calculate a total serious illness score for each individual based on the functional scores and store the total serious illness score at a serious illness score database, retrieve the total serious illness score and historical serious illness scores for an individual of the population of individuals from the serious illness score database in response to a request for a serious illness assessment of the individual, and determine a serious illness score change for the individual based on the total serious illness score and historical serious illness scores for the individual, automatically assign the individual to a clinical queue of a plurality of clinical queues based on the serious illness score change and the total serious illness score, and automatically update the clinical queue to include the individual, wherein each clinical queue comprises a list of individuals prioritized for healthcare intervention, automatically generate a message comprising the updated clinical queue, and transmit the message to at least one client device associated with a clinician to allow the clinician to review the updated clinical queue and provide recommendations for treatment based on the updated clinical queue. In a first example of the system, the executable program is further configured to cause the computer processor to process medical claims for the individual associated with a time duration to identify one or more claim lines, and generate a functional score for a category of the plurality of categories based on the one or more claim lines. In a second example of the system, optionally including the first example, the executable program is further configured to cause the computer processor to generate the functional score for the category based on the one or more claim lines according to a model configured to convert the one or more claim lines to the functional score for the category. In a third example of the system, optionally including one or both of the first and second examples, determining the serious illness score change comprises measuring changes in each functional score of the functional scores over time for the individual. In a fourth example of the system, optionally including one or more or each of the first through third examples, the executable program is further configured to cause the computer processor to calculate event scores for the individual based on ongoing health events and historical health events, and calculate the total serious illness score based on the event scores and the functional scores. In a fifth example of the system, optionally including one or more or each of the first through fourth examples, the plurality of categories includes ambulation, conscious level, activity and evidence of disease, food intake, self-care ability, and obstacles to care. In a sixth example of the system, optionally including one or more or each of the first through fifth examples, the executable program is further configured to cause the computer processor to assign the individual to a position within the clinical queue based on the total serious illness score.

The disclosure also provides support for a system, comprising: a client device configured for a user, and a server communicatively coupled to the client device, the server configured with executable instructions in non-transitory memory of the server that when executed cause a processor of the server to: store healthcare data for a population of individuals in a plurality of network-based databases according to a plurality of categories, convert the healthcare data to functional scores of the plurality of categories for each individual of the population of individuals, calculate a total serious illness score for each individual based on the functional scores and store the total serious illness score at a serious illness score database, retrieve the total serious illness score and historical serious illness scores for an individual of the population of individuals from the serious illness score database in response to a request for a serious illness assessment of the individual, and determine a serious illness score change for the individual based on the total serious illness score and historical serious illness scores for the individual, automatically assign the individual to a clinical queue of a plurality of clinical queues based on the serious illness score change and the total serious illness score, and automatically update the clinical queue to include the individual, wherein each clinical queue comprises a list of individuals prioritized for healthcare intervention, automatically generate a message comprising the updated clinical queue, and transmit the message to at least one client device associated with a clinician to allow the clinician to review the updated clinical queue and provide recommendations for treatment based on the updated clinical queue. In a first example of the system, the server is further configured with executable instructions in non-transitory memory of the server that when executed cause the processor of the server to process medical claims for the individual associated with a time duration to identify one or more claim lines, and generate a functional score for a category of the plurality of categories based on the one or more claim lines. In a second example of the system, optionally including the first example, the server is further configured with executable instructions in non-transitory memory of the server that when executed cause the processor of the server to generate the functional score for the category based on the one or more claim lines according to a model configured to convert the one or more claim lines to the functional score for the category. In a third example of the system, optionally including one or both of the first and second examples, the serious illness score change is determined by measuring changes in each functional score of the functional scores over time for the individual. In a fourth example of the system, optionally including one or more or each of the first through third examples, the server is further configured with executable instructions in non-transitory memory of the server that when executed cause the processor of the server to calculate event scores for the individual based on ongoing health events and historical health events, and calculate the total serious illness score based on the event scores and the functional scores. In a fifth example of the system, optionally including one or more or each of the first through fourth examples, the server is further configured with executable instructions in non-transitory memory of the server that when executed cause the processor of the server to assign the individual to a position within the clinical queue based on the total serious illness score.

As used herein, an element or step recited in the singular and preceded with the word “a” or “an” should be understood as not excluding plural of said elements or steps, unless such exclusion is explicitly stated. Furthermore, references to “one embodiment” of the present disclosure are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the present disclosure, including making and using any devices or systems and performing any incorporated methods. The patentable scope of the present disclosure is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
storing healthcare data for a population of individuals in a plurality of network-based databases according to a plurality of categories;
converting, with a processor configured with a serious illness score module, the healthcare data to functional scores of the plurality of categories for each individual of the population of individuals;
calculating, with the processor, a total serious illness score for each individual based on the functional scores and storing the total serious illness score at a serious illness score database of the serious illness score module;
retrieving, with the processor, the total serious illness score and historical serious illness scores for an individual of the population of individuals from the serious illness score database, in response to a request for a serious illness assessment of the individual, and determining a serious illness score change for the individual based on the total serious illness score and historical serious illness scores for the individual;
automatically assigning, with a queue module of the processor, the individual to a clinical queue of a plurality of clinical queues based on the serious illness score change and the total serious illness score, and automatically updating the clinical queue to include the individual, wherein each clinical queue comprises a list of individuals prioritized for healthcare intervention;
automatically generating, with the queue module, a message comprising the updated clinical queue; and
transmitting, with the processor, the message to at least one client device associated with a clinician to allow the clinician to review the updated clinical queue and provide recommendations for treatment based on the updated clinical queue.

2. The method of claim 1, wherein converting the healthcare data to the functional scores of the plurality of categories comprises processing medical claims for the population of individuals associated with a time duration to identify one or more claim lines, and generating a functional score for a category of the plurality of categories based on the one or more claim lines.

3. The method of claim 2, further comprising generating the functional score for the category based on the one or more claim lines according to a model configured to convert the one or more claim lines to the functional score for the category.

4. The method of claim 1, wherein determining the serious illness score change comprises measuring changes in each functional score of the functional scores over time for the individual.

5. The method of claim 1, further comprising calculating event scores for the individual based on ongoing health events and historical health events, and calculating the total serious illness score based on the event scores and the functional scores.

6. The method of claim 1, wherein the plurality of categories includes ambulation, conscious level, activity and evidence of disease, food intake, self-care ability, and obstacles to care.

7. The method of claim 1, wherein automatically updating the clinical queue to include the individual based on the serious illness score change and the total serious illness score includes assigning the individual to a position within the clinical queue based on the total serious illness score.

8. A computer-readable storage medium including an executable program stored thereon, the executable program configured to cause a computer processor to:
store healthcare data for a population of individuals in a plurality of network-based databases according to a plurality of categories;
convert the healthcare data to functional scores of the plurality of categories for each individual of the population of individuals;
calculate a total serious illness score for each individual based on the functional scores and store the total serious illness score at a serious illness score database;
retrieve the total serious illness score and historical serious illness scores for an individual of the population of individuals from the serious illness score database in response to a request for a serious illness assessment of the individual, and determine a serious illness score change for the individual based on the total serious illness score and historical serious illness scores for the individual;
automatically assign the individual to a clinical queue of a plurality of clinical queues based on the serious illness score change and the total serious illness score, and automatically update the clinical queue to include the individual, wherein each clinical queue comprises a list of individuals prioritized for healthcare intervention;
automatically generate a message comprising the updated clinical queue; and
transmit the message to at least one client device associated with a clinician to allow the clinician to review the updated clinical queue and provide recommendations for treatment based on the updated clinical queue.

9. The computer-readable storage medium of claim 8, wherein the executable program is further configured to cause the computer processor to process medical claims for the individual associated with a time duration to identify one or more claim lines, and generate a functional score for a category of the plurality of categories based on the one or more claim lines.

10. The computer-readable storage medium of claim 9, wherein the executable program is further configured to cause the computer processor to generate the functional score for the category based on the one or more claim lines according to a model configured to convert the one or more claim lines to the functional score for the category.

11. The computer-readable storage medium of claim 8, wherein determining the serious illness score change comprises measuring changes in each functional score of the functional scores over time for the individual.

12. The computer-readable storage medium of claim 8, wherein the executable program is further configured to cause the computer processor to calculate event scores for the individual based on ongoing health events and historical health events, and calculate the total serious illness score based on the event scores and the functional scores.

13. The computer-readable storage medium of claim 8, wherein the plurality of categories includes ambulation, conscious level, activity and evidence of disease, food intake, self-care ability, and obstacles to care.

14. The computer-readable storage medium of claim 8, wherein the executable program is further configured to cause the computer processor to assign the individual to a position within the clinical queue based on the total serious illness score.

15. A system, comprising:
a client device configured for a user; and
a server communicatively coupled to the client device, the server configured with executable instructions in non-transitory memory of the server that when executed cause a processor of the server to:
store healthcare data for a population of individuals in a plurality of network-based databases according to a plurality of categories;
convert the healthcare data to functional scores of the plurality of categories for each individual of the population of individuals;
calculate a total serious illness score for each individual based on the functional scores and store the total serious illness score at a serious illness score database;
retrieve the total serious illness score and historical serious illness scores for an individual of the population of individuals from the serious illness score database in response to a request for a serious illness assessment of the individual, and determine a serious illness score change for the individual based on the total serious illness score and historical serious illness scores for the individual;
automatically assign the individual to a clinical queue of a plurality of clinical queues based on the serious illness score change and the total serious illness score, and automatically update the clinical queue to include the individual, wherein each clinical queue comprises a list of individuals prioritized for healthcare intervention;
automatically generate a message comprising the updated clinical queue; and
transmit the message to at least one client device associated with a clinician to allow the clinician to review the updated clinical queue and provide recommendations for treatment based on the updated clinical queue.

16. The system of claim 15, wherein the server is further configured with executable instructions in non-transitory memory of the server that when executed cause the processor of the server to process medical claims for the individual associated with a time duration to identify one or more claim lines, and generate a functional score for a category of the plurality of categories based on the one or more claim lines.

17. The system of claim 16, wherein the server is further configured with executable instructions in non-transitory memory of the server that when executed cause the processor of the server to generate the functional score for the category based on the one or more claim lines according to a model configured to convert the one or more claim lines to the functional score for the category.

18. The system of claim 15, wherein the serious illness score change is determined by measuring changes in each functional score of the functional scores over time for the individual.

19. The system of claim 15, wherein the server is further configured with executable instructions in non-transitory memory of the server that when executed cause the processor of the server to calculate event scores for the individual based on ongoing health events and historical health events, and calculate the total serious illness score based on the event scores and the functional scores.

20. The system of claim 15, wherein the server is further configured with executable instructions in non-transitory memory of the server that when executed cause the processor of the server to assign the individual to a position within the clinical queue based on the total serious illness score.
